(19) United States
(12) Patent Application Publication
(10) Pub. No.: US 2016/0074309 A1
(43) Pub. Date: Mar. 17, 2016

(54) COSMETIC SKIN-CARE AGENT

(71) Applicant: Henkel AG & Co. KGaA, Duesseldorf (DE)

(72) Inventors: Daniela Kessler-Becker, Leverkusen (DE); Thomas Foerster, Duesseldorf (DE); Markus Semrau, Langenfeld (DE)

(21) Appl. No.: 14/951,901

(22) Filed: Nov. 25, 2015

Related U.S. Application Data
(63) Continuation of application No. PCT/EP2014/052016, filed on Feb. 3, 2014.

(30) Foreign Application Priority Data
May 28, 2013 (DE) 10 2013 209 896.0

(51) Int. Cl. A61K 8/85; A61Q 19/08; A61K 8/97; A61K 8/34; A61K 8/67; A61K 8/64

(52) U.S. Cl. CPC A61K 8/85; A61K 8/678; A61K 8/64; A61K 8/97; A61K 8/345; A61Q 19/08; A61K 2800/412

(57) ABSTRACT

Cosmetic or dermatological topical compositions include, in a suitable cosmetic or dermatological carrier, a) polylactic acid particles and b) at least one active substance selected from monomers, oligomers, and polymers of amino acids, N—C₂-C₂₄ acylamino acids, the esters and/or physiologically acceptable metal salts of said substances and/or—extracts from the group comprising Quassia amara, dill (Peucedanum graveolens), currant, cardamom (Elettaria cardamomum), black radish, butcher's broom, cinnamon, oats (Avena sativa), potato, silk, and asafoetida gum and/or—hyaluronic acid and/or—apple seed extract and/or—phytohormones and/or—isoflavonoids and/or—phytosterols and/or—triterpenoids and/or—tocopherols and/or—6,7-disubstituted 2,2-dialkyl chromanes or chromenes of general formula (II) or (III) disclosed herein.

### FIELD OF THE INVENTION

The present invention generally relates to topical cosmetic or dermatological compositions for skin treatment.

### BACKGROUND OF THE INVENTION

The structure and properties of human skin change due to age. The aging process is characterized by a progressive transition of skin cells from a proliferative state to a quiescent, senescent state. This transition corresponds to aging at the cellular level and most of the macroscopic aging effects can be attributed to it. Visible signs are increased wrinkle formation, which is accompanied by a change in the top and bottom skin layers at the microscopic level. Aged skin, moreover, is said to be especially susceptible to external stressors such as UV light.

Consumers with mature skin in particular desire a smooth complexion with simultaneous wrinkle reduction.

The typical signs of skin aging, such as wrinkles and dry skin, are based on biological processes such as an altered regulation of cell physiology and a reduced energy supply to skin cells, as well as a constantly reforming horny layer (skin barrier). To counteract these processes associated with skin aging, it is helpful to stimulate the renewal processes in the top skin layers (epidermis) and to promote the building of connective tissue in the deeper skin layers (dermis). Various cosmetic bioactive substances can be used for this purpose.

It is therefore desirable to provide polylactate particles with specific nurturing active substances that together impart high skin care and a skin regenerating effect. Suitable active substance combinations would increase skin moisture and counteract the typical signs of aging.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Cosmetic or dermatological topical compositions include, in a suitable cosmetic or dermatological carrier, a) polylactic acid particles and b) at least one active substance from the group comprising: monomers, oligomers, and polymers of amino acids, N—C₂-C₂₄ acylamino acids, the esters and/or physiologically acceptable metal salts of said substances; and/or plant preparations, especially expressed juices and extracts which are obtained from plants in the group comprising Quassia amara, dill (Peucedanum graveolens), currant, cardamom (Elettaria cardamomum), black radish, butcher's broom, cinnamon, oats (Avena sativa), potato, silk, and asafoetida gum; and/or hyaluronic acid; and/or apple seed extract; and/or phytohormones; and/or iso flavonoids; and/or phytosterols; and/or triterpenoids; and/or tocopherols; and/or 6,7-disubstituted 2,2-dialkyl chromanes or chromenes of general formula (II) or (III),

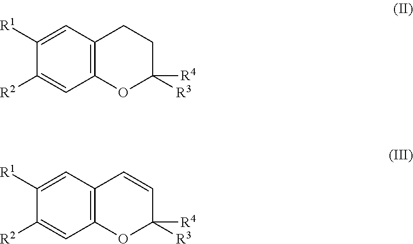

R¹ and R² representing, independently of one another, an OH group, a methoxy group, or a CF₃CH₂O group and R³ and R⁴ representing, independently of one another, a C₁-C₄ alkyl group, in particular lipochroman-6; and/or ellagic acid and/or ellagates; and/or xanthophylls, in particular astaxanthin; and/or superoxide dismutases; and/or fermentations based on lactic acid bacteria; and/or ethyl hexanoate and derivatives thereof; and/or methyl butyrate and derivatives thereof; and/or ethyl decadienoate and derivatives thereof.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject of the present application is cosmetic or dermatological topical compositions including, in a suitable cosmetic or dermatological carrier,
a) polylactic acid particles and
b) at least one active substance from the group comprising
   monomers, oligomers, and polymers of amino acids, N—C₂-C₂₄ acylamino acids, the esters and/or physiologically acceptable metal salts of said substances and/or
   plant preparations, especially expressed juices and extracts which are obtained from plants in the group comprising Quassia amara, dill (Peucedanum graveolens), currant, cardamom (Elettaria cardamomum), black radish, butcher's broom, cinnamon, oats (Avena sativa), potato, silk, and asafoetida gum, and/or
   hyaluronic acid and/or
   apple seed extract and/or
   phytohormones and/or
   isoflavonoids and/or
   phytosterols and/or
   triterpenoids and/or
   tocopherols and/or
   6,7-disubstituted 2,2-dialkyl chromanes or chromenes of general formula (II) or (III),

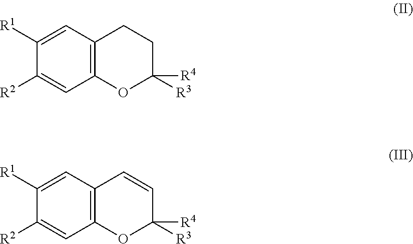

R₁ and R² representing, independently of one another, an OH group, a methoxy group, or a CF₃CH₂O group and R³ and R⁴ representing, independently of one another, a C₁-C₄ alkyl group, in particular lipochroman-6 and/or
ellagic acid and/or ellagates and/or
xanthophylls, in particular astaxanthin, and/or
superoxide dismutases and/or
fermentations based on lactic acid bacteria and/or
ethyl hexanoate and derivatives thereof and/or
methyl butyrate and derivatives thereof and/or
ethyl decadienoate and derivatives thereof.

Polylactic acid particles a) are a first essential component of the cosmetic compositions of the invention. The use of polylactic acid particles in cosmetics is known, for example, from the international patent applications WO 2012/177615 A1 and WO 2012/177617 A1. The percentage by weight of the polylactic acid particles a) of the total weight of the cosmetic compositions according to the invention is preferably 0.1 to 20% by weight, preferably 0.5 to 15% by weight, especially preferably 0.75 to 10% by weight, and especially 1 to 8% by weight.

Polylactic acid, also called polylactide or PLA, is a name for biodegradable polymers (polyesters), which are obtainable primarily by the ionic polymerization of lactide, a ring-shaped joining of two lactic acid molecules.

A ring-opening polymerization occurs at temperatures between 140 and 180° C. and under the effect of catalytic tin compounds (e.g., tin oxide). Thus, plastics with a high molecular weight and strength are produced. Lactide itself can be produced by fermentation of molasses or by fermentation of glucose with the aid of various bacteria. Moreover, high-molecular-weight and pure polylactides can be produced directly from lactic acid with the aid of so-called polycondensation. Nevertheless, the disposal of the solvent is problematic in industrial production.

Lactic acid (2-hydroxypropanoic acid) has an asymmetric C atom, so that polylactic acid as well has optically active centers in the L(+) and D(−) configuration. The ratio of L- to D-monomer units in this regard determines the degree of crystallinity, the melting point, and the biodegradability of the polymers.

Polylactic acids suitable according to the invention are L-polylactic acid, D-polylactic acid, and L/D-polylactic acid, and mixtures thereof L-polylactic acid is especially preferred because of its very good biodegradability. In a preferred embodiment of the present invention, the percentage by weight of the L-lactic acid monomer units in the polylactic acid is greater than 50% by weight, preferably greater than 80% by weight, and especially greater than 90% by weight.

The molar mass of the polylactic acid suitable according to the invention is preferably 1000 to 1,000,000, preferably 10,000 to 300,000, more preferably 50,000 to 250,000, and especially 100,000 to 180,000 daltons.

In another preferred embodiment of the present invention, polylactic acid is used in a form blended with fillers. The use of greater filler amounts is helpful in reducing the polymer into particles and increases the biodegradability and the inner specific surface via porosity and capillarity. In this case, water-soluble fillers are particularly preferred, for example, metal chlorides such as NaCl, KCl, etc., metal carbonates such as Na₂CO_{3,} NaHCO_{3,} etc., and metal sulfates such as MgSO₄.

Natural raw materials can also be used as fillers, for example, nut shells, wood or bamboo fibers, starch, xanthan gum, alginates, dextran, agar etc. These fillers are biodegradable and do not cause the good ecological properties of polylactic acid particles to worsen. The content of biodegradable fillers in the polylactic acid particles can be typically 10 to 70% by weight, whereby amounts of 20 to 60% by weight are preferred and those of 30 to 50% by weight are especially preferred.

Polylactic acid particles suitable according to the invention can be present both as spherical and as irregular particles, which have a specific circularity.

It is assumed that irregular shapes can intensify the abrasiveness of the polylactic acid particles; therefore, it can be advantageous for some embodiments of the present invention if the polylactic acid particles preferably have a circularity between 0.1 and 0.6. Polylactic acid particles with a lower circularity, in contrast, can be preferred if a less abrasive, gentler abrading action of the cleaning agent according to the invention is to be achieved.

The shape of the polylactic acid particles employed according to the invention can be defined in various ways, whereby within the scope of this preferred embodiment of the present invention the geometric proportions of a particle and, more pragmatically, of a particle population, are determined.

More recent, highly precise methods permit the precise determination of particle shapes from a large number of particles, typically of more than 10,000 particles, preferably of more than 100,000 particles. These methods enable a precise selection of the average particle shape of a particle population. The determination of particle shapes is preferably carried out with an “Occhio Nano 500 Particle Characterisation Instrument” with the software “Callistro version 25” (Occhio s.a. Liege, Belgium). This instrument enables the preparation, dispersing, imaging, and analysis of a particle population, whereby preferably the instrument parameters are set as follows: White Requested=180, vacuum time=5000 ms, sedimentation timer=5000 ms, automatic threshold, number of particles counted/analyses=8000 to 500,000, minimum number of replicates/sample=3, lens setting 1×/1.5×.

The polylactic acid particles, used according to the invention, preferably have sizes defined by their area-equivalent diameter (ISO 9276-6: 2008(E) Section 7), also called “Equivalent Circle Diameter ECD” (ASTM F1877-05 Section 11.3.2). The mean ECD of a particle population is calculated as the mean ECD of each individual particle of a particle population of at least 10,000 particles, preferably of more than 50,000 particles, especially of more than 100,000 particles, after particles with an area-equivalent diameter (ECD) below 10 μm were excluded from the measurement. In a preferred embodiment of the present invention, the polylactic acid particles have mean ECD values of 10 to 1000 μm, preferably of 50 to 500 μm, more preferably of 100 to 350 μm, and especially of 150 to 250 μm.

Independent of the average particle size, cosmetic cleaning agents according to the invention are preferred in which the polylactic acid particles have absolute particle sizes of 1 to 1000 μm, more preferably of 1 to 850 μm, particularly preferably of 1 to 750 μm, exceptionally preferably of 1 to 500 μm, and especially preferably of 1 to 300 μm.

Within the scope of the present invention, shape descriptors are used which are calculations of geometric descriptors or shape factors. Shape factors are ratios between two different geometric properties, which for their part are a measurement of the proportions of the image of a whole particle or the measurement of the proportions of an ideal geometric body, enveloping the particle.

These results are descriptors similar to size ratios (aspect ratios). In a preferred embodiment of the present invention, mesoshape descriptors are used for particle characterization. These mesoshape descriptors indicate the extent to which a particle deviates from an ideal geometric shape, particularly from a sphere.

In a first preferred embodiment of the present invention, the polylactic acid particles can deviate from the typical spherical shape or sphere-like shapes such as, for example, granular particles (see above). In this case, the particles preferably have sharp corners and edges and preferably possess concave curvatures. Sharp corners of non-spherical particles in this regard are defined by a radius less than 20 μm, preferably less than 8 μm, and especially less than 5 μm, whereby the radius is defined as the radius of an imaginary circle that follows the contour of the corner.

Circularity is a quantitative, 2-dimensional image analysis and can be determined according to ISO 9276-6: 2008(E) Section 8.2. Circularity is a preferred mesoshape descriptor and can be determined, for example, with the above-described “Occhio Nano 500 Particle Characterisation Instrument” with the software “Callistro version 25” (Occhio s.a. Liege, Belgium) or with the “Malvern Morphologi G3.” Circularity is occasionally described in the literature as the difference between a particle and the perfect spherical shape. The values for circularity vary between 0 and 1, whereby 1 describes the perfect sphere or (in the two-dimensional image) the perfect circle:

C=[(4πA(/p²]^{1/2}

where A is the projection area (the two-dimensional descriptor) and p the length of the perimeter of the particle.

Within said preferred embodiment, polylactic acid particles with a mean circularity C of 0.1 to 0.6, preferably of 0.15 to 0.4, and especially of 0.2 to 0.35 have proven especially suitable within the scope of the present invention. In this case, the mean values are obtained by quotient formation from volume-based measurements and number-based measurements.

Solidity is a quantitative, 2-dimensional image analysis and can be determined according to ISO 9276-6: 2008(E) Section 8.2. Solidity is likewise a preferred mesoshape descriptor and can be determined, for example, with the above-described “Occhio Nano 500 Particle Characterisation Instrument” with the software “Callistro version 25” (Occhio s.a. Liege, Belgium) or with the “Malvern Morphologi G3.” Solidity is a mesoshape descriptor, which describes the concavity of a particle or a particle population. Solidity values vary between 0 and 1, whereby a solidity number of 1 describes a non-concave particle:

Solidity=A/Ac

where A is the (image) area of the particle and Ac is the area of the convex shell enveloping the particle.

Within the first preferred embodiment, polylactic acid particles having a mean solidity of 0.4 to 0.9, preferably of 0.5 to 0.8, and especially of 0.55 to 0.65 have proven to be especially suitable within the scope of the invention. In this case, the mean values are obtained by quotient formation from volume-based measurements and number-based measurements.

Especially preferred polylactic acid particles of the first preferred embodiment preferably have a mean circularity C of 0.1 to 0.6, preferably of 0.15 to 0.4, and especially of 0.2 to 0.35 and a mean solidity of 0.4 to 0.9, preferably of 0.5 to 0.8, and especially of 0.55 to 0.65.

“Mean” circularity and solidity are averages from the measurement of a large number of particles, typically of more than 10,000 particles, preferably of more than 50,000 particles, and especially of more than 100,000 particles, whereby particles with an area-equivalent diameter (ECD) of less than 10 μm were excluded from the measurement.

After its preparation, the polylactic acid polymer can be converted to the desired particle size and shape, for example, by a grinding process, depending on the shape required for the particular purpose.

An especially preferred method for preparing polylactic acid particles with the desired circularity and solidity consists of preparing a foam from polylactic acid and subsequent grinding.

It is assumed that a specific hardness can enhance the abrasive effect of the polylactic acid particles; therefore, it can be advantageous for some embodiments of the present invention, furthermore, if the polylactic acid particles have hardnesses of 3 to 50 kg/mm², preferably of 4 to 25 kg/mm², and especially of 5 to 15 kg/mm² on the HV Vickers hardness scale.

The hardness of the particles in this case can be varied via the ratio of the D- to L-monomers and via the molar mass.

Polylactic acid particles, which can be used preferably in the cleaning agents of the invention, are commercially available (for example, from the company Micro Powders, Inc., under the trade name Ecosrub®). Especially preferred are the commercial products Ecosrub® 20PC, Ecosrub® 50PC, Ecosrub® 100PC, Ecoblue® 5025, and Ecogreen® 5025. Preferred in particular are Ecosrub® 20PC and Ecosrub® 50PC.

The compositions of the invention include a nurturing “anti-aging” active substance b) as the second essential ingredient. Compositions preferred according to the invention include, based on their total weight, a percentage by weight of 0.001 to 10% by weight, preferably 0.01 to 7.5% by weight, especially preferably 0.05 to 5% by weight, and especially 0.1 to 3% by weight of active substance b).

Cosmetic compositions preferred according to the invention are characterized in that, based on their weight, they include
a) 0.1 to 20% by weight, preferably 0.5 to 15% by weight, especially preferably 0.75 to 10% by weight, and especially 1 to 8% by weight of polylactic acid particles, and
b) 0.001 to 10% by weight, preferably 0.01 to 7.5% by weight, especially preferably 0.05 to 5% by weight, and especially 0.1 to 3% by weight of at least one active substance from group b).

Cosmetic compositions have proven especially advantageous in regard to the nurturing and rejuvenating effect, said compositions in which active substance b) is selected from the group comprising
oligomers of amino acids, N—C₂-C₂₄ acylamino acids, and/or the physiologically acceptable metal salts of said substances and/or
extracts from the leaves, bark, and/or wood of Quassia amara.

Corresponding cosmetic compositions are preferred according to the invention.

Especially preferred oligomers of amino acids, N—C₂-C₂₄ acyl amino acids, and/or the physiologically acceptable metal salts of said substances are selected from di-, tri-, tetra-, penta-, hexa-, or pentadecapeptides, which may be N-acylated and/or esterified, especially preferably from

Tyr-Arg and its N-acylated derivatives, particularly N-acetyl-Tyr-Arg hexyldecyl esters,

Gly-His-Lys and its N-acylated derivatives, particularly N-palmitoyl-Gly-His-Lys,

Gly-His-Arg and its N-acylated derivatives, particularly N-myristoyl-Gly-His-Arg,

Lys-Val-Lys and its N-acylated derivatives, particularly palmitoyl-Lys-Val-Lys,

Val-Tyr-Val,

Gly-Gln-Pro-Arg (rigin), rigin analogues, and rigin derivatives, particularly N-palmitoyl-Gly-Gln-Pro-Arg,

Lys-Thr-Thr-Lys-Ser and its N-acylated derivatives, particularly N-palmitoyl-Lys-Thr-Thr-Lys-Ser,

Val-Gly-Val-Ala-Pro-Gly and its N-acylated derivatives, particularly N-palmitoyl-Val-Gly-Val-Ala-Pro-Gly,

acetyl hexapeptide-3, hexapeptide-4, hexapeptide-5, myristoyl hexapeptide-5, myristoyl hexapeptide-6, hexapeptide-8, hexapeptide-9, hexapeptide-10, L-glutamylaminoethyl indole,

and combinations of said substances, especially combinations of N-palmitoyl-Gly-His-Lys and N-palmitoyl-Gly-Gln-Pro-Arg.

Plant preparations, especially expressed juices and extracts, of Quassia amara are used with particular advantage as active substance b). The expressed juices or extracts are preferably obtained from the herbaceous parts (the aboveground plant parts) and/or roots of Quassia amara. The expressed juices are obtained in a preferable manner by mechanical pressing.

An extract within the meaning of the present application is a substance or substance mixture that was obtained by extraction and partial or complete evaporation of the extraction solution. The following are differentiated according to their nature: dry extracts, i.e., extracts evaporated to dryness; fluid extracts, i.e., extracts produced with solvents so that at most 2 parts of fluid extract are recovered from one part drug; viscous extracts or thick extracts, i.e., extracts in which a portion of the solvent is evaporated.

The extracts or expressed juices used according to the invention can be obtained from freshly harvested plants but also from stored products.

The extracts can be produced with water, as well as with polar or nonpolar organic solvents, as well as with mixtures thereof in a manner known to the skilled artisan. The extracts used according to the invention are obtained by extraction preferably with organic solvents, water, or mixtures thereof. Preferably suitable organic solvents are ketones (e.g., acetone), ethers, esters, alcohols, or halogenated hydrocarbons. Especially preferred extracting agents are water and/or alcohols. Of the alcohols, (C₁ to C₆) alcohols, such as ethanol and isopropanol, namely both as the only extracting agent and also in a mixture with water, are preferred in this case. Especially preferred extracting agents are water, ethanol, 2-propanol, 1,2-propylene glycol, 1,3-butylene glycol; very especially preferred are water, ethanol, 2-propanol, and 1,2-propylene glycol, and mixtures thereof, e.g., a mixture of 1,2-propylene glycol/water in a 4:1 ratio. Extracts, which can be obtained by extraction with ethanol or water/ethanol mixtures, and expressed juice are especially preferred.

Both the extracts in the original extracting agent and extracts/expressed juice in water or other organic solvents and/or mixtures thereof, particularly ethanol and ethanol/water mixtures, can be used. Preferably, the extracted or pressed material is used as a solid from which the solvent (particularly as gently as possible) was removed. However, it is also possible to use extracts/expressed juices from which the solvent was partially removed, so that a thickened extract/expressed juice is used. Expressed juices from fresh Quassia amara are very especially preferred. The extracts and/or expressed juices are used in particular in solid form. The extraction is carried out preferably at a temperature of 25° C. to 90° C.

It can also be preferred in another embodiment that the active substance mixture to be used according to the invention, preferably the plant preparations, are characterized in that the extract or expressed juice includes at least one polar solvent, selected from ethanol, 1-propanol, 2-propanol, 1-butanol, 2-butanol, ethylene glycol, 1,2-propylene glycol, 1,3-butylene glycol, glycerol, and water, and mixtures thereof.

Depending on the choice of extracting agent, it can be preferable to stabilize the extract by adding a solubilizer. Suitable as solubilizers are, for example, ethoxylation products of optionally hydrogenated vegetable and animal oils. Preferred solubilizers are ethoxylated mono-, di-, and triglycerides of C8-22 fatty acids with 4 to 50 ethylene oxide units, e.g., hydrogenated ethoxylated castor oil, olive oil ethoxylate, almond oil ethoxylate, mink oil ethoxylate, polyoxyethylene glycol caprylic/capric acid glycerides, polyoxyethylene glycerol monolaurate, and polyoxyethylene glycol coconut fatty acid glycerides. Especially preferred is olive oil ethoxylate (INCI name: PEG-10 Olive Glycerides).

The dry mass of the extract or expressed juice depends on the molar mass and solubility of the dispersed ingredients and is usually 1 to 80% by weight, in each case based on the weight of the extract or the expressed juice. Preferably, the dry mass is 15 to 50% by weight and especially preferably 20 to 35% by weight. At a molecular weight of the ingredients of over 100,000 daltons, a dry mass of 1 to 20% by weight can be preferred and a dry mass of 1 to 10% by weight can be especially preferred. Especially preferred cosmetic agents are characterized in that the dry mass of the extract or the expressed juice is 5 to 80% by weight, based on the weight of the extract or expressed juice.

The compositions of the invention include the active substance combination a) and b) in a suitable cosmetic carrier. This is taken to mean preferably a topical carrier, which is preferably an aqueous or aqueous-alcoholic carrier. The carrier preferably includes, based on its total weight, at least 20% by weight, more preferably at least 25% by weight, especially preferably at least 30% by weight, and especially at least 35% by weight of water. Especially preferred cosmetic compositions are characterized by a water content between 30 and 90% by weight, preferably 40 to 80% by weight, especially preferably 50 to 70% by weight, and especially 55 to 65% by weight.

Furthermore, the cosmetic carrier can include 0.01 to 40% by weight, preferably 0.05 to 35% by weight, and especially 0.1 to 30% by weight of at least one alcohol, which can be selected from ethanol, 1-propanol, 2-propanol, isopropanol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol, phenoxyethanol, or mixtures said alcohols. The water-soluble alcohols are preferred.

Ethanol, 1-propanol, 2-propanol, 1,2-propylene glycol, glycerol, and/or 1,6-hexanediol, and mixtures of said alcohols are especially preferred. Glycerol and/or 1,6-hexanediol are especially preferred.

The use of polyols has proven especially advantageous for the cosmetic effect of compositions of the invention. Preferred cosmetic compositions include 0.1 to 20% by weight, preferably 0.5 to 15% by weight, especially preferably 1 to 10% by weight, and especially 2 to 8% by weight of at least one polyol, selected from glycerol, 1,2-propylene glycol, 1,3-butylene glycol, and 1,6-hexanediol, whereby the quantitative data refer to the weight of the cosmetic compositions.

Emulsions especially suitable according to the invention include, in addition to the already described active substances, preferably at least one additional skin-conditioning active substance c) and/or at least one emulsifier d).

Suitable skin-conditioning active substances c) are to be taken to mean preferably substances that are absorbed onto keratinic materials, particularly onto the skin, and improve the physical and sensory properties of both the skin and the product as such. Conditioning agents smooth the topmost layer of the skin and make it soft and supple. The skin feel of the entire product can be adjusted via the selection of the conditioning agents (oily-less oily, rapidly or slowly spreading, rapidly or slowly absorbed into the skin, and so forth).

Preferred skin-conditioning active substances c) can be selected from fatty substances, particularly plant oils, such as sunflower oil, olive oil, soybean oil, rapeseed oil, almond oil, jojoba oil, orange oil, wheat germ oil, peach kernel oil, and the liquid fractions of coconut oil, lanolin, and the derivatives thereof, liquid paraffin oils, isoparaffin oils, and synthetic hydrocarbons, di-n-alkyl ethers having a total of 12 to 36 C atoms, e.g., di-n-octyl ether and n-hexyl n-octyl ether, fatty acids, especially linear and/or branched, saturated and/or unsaturated C₈₋₃₀ fatty acids, fatty alcohols, especially saturated, mono- or polyunsaturated, branched or unbranched fatty alcohols having 4-30 carbon atoms, which can be ethoxylated with 1-75, preferably 5-20 ethylene oxide units, and/or propoxylated with 3-30, preferably 9-14 propylene oxide units, ester oils, i.e., esters of C₆₋₃₀ fatty acids with C₂₋₃₀ fatty alcohols, hydroxycarboxylic acid alkyl esters, dicarboxylic acid esters such as di-n-butyl adipate, and diol esters such as ethylene glycol dioleate or propylene glycol di(2-ethylhexanoate), symmetric, asymmetric, or cyclic esters of carbonic acid with fatty alcohols, e.g., glycerol carbonate or dicaprylyl carbonate (Cetiol® CC), mono, di-, and trifatty acid esters of saturated and/or unsaturated linear and/or branched fatty acids with glycerol, which can be ethoxylated with 1-10, preferably 7-9 ethylene oxide units, e.g., PEG-7 glyceryl cocoate, waxes, particularly insect waxes, vegetable waxes, fruit waxes, ozokerite, microwaxes, ceresin, paraffin waxes, triglycerides of saturated and optionally hydroxylated C₁₆₋₃₀ fatty acids, e.g., hydrogenated triglyceride fats, phospholipids, for example, soya lecithin, egg lecithin, and cephalins, shea butter, silicone compounds, selected from decamethylcyclopentasiloxane, dodecamethylcyclohexasiloxane, and silicone polymers, which can be crosslinked if so desired, e.g., polydialkylsiloxanes, polyalkylarylsiloxanes, ethoxylated and/or propoxylated polydialkylsiloxanes with the former INCI name Dimethicone Copolyol, and polydialkylsiloxanes, including amine and/or hydroxy groups, preferably substances with the INCI names Dimethiconol, Amodimethicone, or Trimethylsilylamodimethicone.

In a preferred embodiment, the compositions of the invention include a mixture of skin-conditioning active substances from the aforementioned list.

The use amount of skin-conditioning active substances c) in the compositions of the invention is preferably 1 to 50% by weight, preferably 3 to 40% by weight, especially preferably 5 to 50% by weight, and especially 10 to 30% by weight of at least one skin-conditioning active substance c), whereby the quantitative data refer to the weight of the cosmetic compositions.

Suitable surface-active substances and/or emulsifiers d) are, for example, adducts of 4 to 30 mol of ethylene oxide and/or 0 to 5 mol of propylene oxide to linear C₈-C₂₂ fatty alcohols, to C₁₂-C₂₂ fatty acids, and to C₈-C₁₅ alkylphenols, C₁₂-C₂₂ fatty acid mono- and diesters of adducts of 1 to 30 mol of ethylene oxide to C₃-C₆ polyols, particularly to glycerol, ethylene oxide, and polyglycerol adducts to methylglucoside fatty acid esters, fatty acid alkanolamides, and fatty acid glucamides, C₈-C₂₂ alkyl mono- and oligoglycosides, and the ethoxylated analogues thereof, whereby degrees of oligomerization of 1.1 to 5, especially 1.2 to 2.0, and glucose as the sugar component are preferred, e.g., the product known under the INCI name Coco Glucoside, mixtures of alkyl (oligo) glucosides and fatty alcohols, e.g., the commercially available products Montanov® 68 and Montanov® 202, adducts of 5 to 60 mol of ethylene oxide to castor oil and hydrogenated castor oil, partial esters of polyols having 3-6 carbon atoms with saturated C₈-C₂₂ fatty acids, sterols, particularly cholesterol, lanosterol, beta-sitosterol, stigmasterol, campesterol, and ergosterol, as well as mycosterols, phospholipids, primarily glucose phospolipids, fatty acid esters of sugars and sugar alcohols such as sorbitol, polyglycerols, and polyglycerol derivatives, preferably polyglyceryl-2-dipolyhydroxystearate (commercial product Dehymuls® PGPH) and polyglyceryl-3-diisostearate (commercial product Lameform® TGI), as well as linear and branched C₈-C₃₀ fatty acids and the Na, K, ammonium, Ca, Mg, and Zn salts thereof.

The use of non-ethoxylated anionic O/W emulsifiers is especially preferred. An O/W emulsifier system especially preferred according to the invention comprises a mixture of dipotassium monocetyl phosphate and potassium dicetyl phosphate with hydrogenated palm oil glycerides. A suitable mixture is available as the commercial product “Emulsiphos 677660” (INCI: Potassium Cetyl Phosphate, Hydrogenated Palm Glycerides) from the company Symrise. Especially preferred compositions of the invention include, based on their total weight, 0.1 to 3.0% by weight, preferably 0.2 to 2.0% by weight, and especially 0.5 to 1.5% by weight of a non-ethoxylated anionic O/W emulsifier, preferably a mixture of dipotassium monocetyl phosphate and potassium dicetyl phosphate with hydrogenated palm oil glycerides.

The compositions of the invention can include the emulsifiers d) preferably in amounts of 0.1 to 25% by weight, especially preferably of 0.5 to 15% by weight, and especially of 1 to 10% by weight, based on the total composition.

Other cosmetic compositions especially preferred according to the invention are characterized in that they include furthermore at least one skin-soothing active substance e). Skin-soothing active substances preferred according to the invention are selected from allantoin, α-bisabolol, α-lipoic acid, extracts of Centella asiatica, for example, obtainable under the name Madecassicoside from DSM, glycyrrhetinic acid, which is especially preferably encapsulated in liposomes and is obtainable in this form, e.g., under the trade name Calmsphere from Soliance, mixtures of grain waxes, extracts from shea butter, and Argania spinosa oil with the INCI name “Spent grain wax and Butyrospermum Parkii (shea butter) extract and Argania Spinosa Kernel Oil,” as they are obainable, e.g., under the trade name Stimu-Tex AS from the company Pentapharm, extracts of Vanilla tahitensis, as they are obtainable, e.g., under the trade name Vanirea (INCI: Vanilla Tahitensis Fruit Extract) from the company Solabia, algin hydrolysates, as they are obtainable, e.g., under the trade name Phycosaccharide, particularly Phycosaccharide AI, from the company Codif, extracts of Bacopa monniera, as they are obtainable, e.g., under the trade name Bacocalmine from the company Sederma, extracts of rooibos plants, as they are obtainable, e.g., under the trade name Rooibos Herbasec MPE from the company Cosmetochem, yeast extracts, especially preferably the commercial product Drieline (INCI name “Sorbitol, Yeast Extract”), obtainable from the company Lanatech, the physiologically acceptable salts of sterol sulfates, as they are obtainable, e.g., under the trade name Phytocohesine (INCI: Sodium Beta-Sitosterylsulfate) from the company Vincience, aminodicarboxylic acids with a C-chain length of 3-6 carbon atoms and the physiologically acceptable salts thereof, preferably selected from aminomalonic acid, aminosuccinic acid (=aspartic acid), aminoglutaric acid, and aminoadipic acid, and the physiologically acceptable salts thereof, such as potassium aspartate and magnesium aspartate, and any mixtures of said substances.

The skin-soothing active substances e) are preferably included in a total amount of 0.001 to 5% by weight, especially preferably 0.01 to 2% by weight, and exceedingly preferably 0.1 to 1% by weight, in each case based on the total composition.

Other suitable additives in emulsions preferred according to the invention and/or aqueous gels are thickeners, e.g., anionic polymers of acrylic acid, methacrylic acid, crotonic acid, maleic anhydride, and 2-acrylamido-2-methylpropanesulfonic acid, whereby the acid groups can be present entirely or partially as the sodium, potassium, ammonium, or mono- or triethanolammonium salts and whereby at least one nonionic monomer can be present. Preferred nonionogenic monomers are acrylamide, methacrylamide, acrylic acid esters, methacrylic acid esters, vinylpyrrolidone, vinyl ethers, and vinyl esters. Preferred anionic copolymers are acrylic acid-acrylamide copolymers and particularly polyacrylamide copolymers with sulfonic acid group-containing monomers. These copolymers can also be present crosslinked. Suitable commercial products are Sepigel® 305, Simulgel® 600, Simulgel® NS, and Simulgel® EPG from the company SEPPIC. Other especially preferred anionic homo- and copolymers are non-crosslinked and crosslinked polyacrylic acids. Such compounds are, for example, the commercial products Carbopol®. An especially preferred anionic copolymer includes as the monomer up to 80-98% of an unsaturated, if desired, substituted C₃₋₆ carboxylic acid or the anhydride thereof and up to 2-20%, if desired, substituted acrylic acid esters of saturated C₁₀₋₃₀ carboxylic acids, whereby the copolymer can be crosslinked with the aforementioned crosslinking agents. Appropriate commercial products are Pemulen® and the Carbopol® types 954, 980, 1342, and ETD 2020 (from B.F. Goodrich).

Another preferred group of ingredients of the compositions of the invention with the active substance complex of the invention are vitamins, provitamins, or vitamin precursors. Vitamins, provitamins, and vitamin precursors, classified in the groups A, B, C, E, F, and H, are especially preferred in this regard.

The group of substances designated as vitamin A includes retinol and 3,4-didehydroretinol. Beta-carotene is the retinol provitamin. Suitable vitamin A components according to the invention are, for example, vitamin A acid and esters thereof, vitamin A aldehyde, and vitamin A alcohol and esters thereof such as the palmitate and the acetate. The agents of the invention include the vitamin A component preferably in amounts of 0.05 to 1% by weight, based on the total preparation.

The vitamin B group or the vitamin B complex includes, inter alia,
Vitamin B-1 (thiamine)
Vitamin B-2 (riboflavin)
Vitamin B-3: The compounds nicotinic acid and nicotinic acid amide (niacinamide) are often included under this term. According to the invention, nicotinic acid amide is preferred, which is included in the agents of the invention preferably in amounts of 0.05 to 1% by weight, based on the total agents.
Vitamin B-5: (pantothenic acid, panthenol, and pantolactone). Panthenol and/or pantolactone are preferably used within the scope of this group. Panthenol derivatives that can be used according to the invention are particularly the esters and ethers of panthenol and cationically derivatized panthenols. Individual representatives are, for example, panthenol triacetate, panthenol monoethyl ether, and the monoacetate thereof, as well as cationic panthenol derivatives. Pantothenic acid is used preferably as a derivative in the form of the more stable calcium salt and sodium salt (Ca pantothenate, Na pantothenate) in the present invention.
Vitamin B-6 (pyridoxine as well as pyridoxamine and pyridoxal).
The aforementioned compounds of the vitamin B type, especially vitamin B-3, B-5, and B-6, are included in the agents of the invention preferably in amounts of 0.05 to 10% by weight, based on the total agent. Amounts of 0.1 to 5% by weight are especially preferred.
Vitamin C (ascorbic acid). Vitamin C is used in the agents of the invention preferably in amounts of 0.1 to 3% by weight, based on the total agent. The use in the form of the palmitic acid ester, glucosides, or phosphates can be preferred. The use in combination with tocopherols can likewise be preferred.
Vitamin E (tocopherols, especially alpha-tocopherol). Tocopherol and its derivatives, which include in particular esters such as acetate, nicotinate, phosphate, and succinate, are included in the agents of the invention preferably in amounts of 0.05 to 1% by weight, based on the total agent.
Vitamin F. The term “vitamin F” is conventionally understood to mean essential fatty acids, in particular linoleic acid, linolenic acid, and arachidonic acid.
Vitamin H. Vitamin H is the name for the compound (3aS,4S,6aR)-2-oxohexahydrothienol[3,4-d]imidazole-4-valeric acid, although the trivial name biotin has for now become accepted. The agents of the invention preferably include biotin in amounts of 0.0001 to 1.0% by weight and especially in amounts of 0.001 to 0.01% by weight.

The compositions of the invention preferably include vitamins, provitamins, and vitamin precursors from the groups A, B, E, and/or H. Panthenol, pantolactone, pyridoxine, and the derivatives thereof, as well as nicotinic acid amide and biotin, are especially preferred.

The cosmetic compositions of the invention are preferably characterized by a high content of natural and/or biodegradable ingredients.

Cosmetic compositions are preferably characterized in that they include 40 to 100% by weight, preferably 45 to 99% by weight, especially preferably 50 to 97% by weight, and especially 60 to 95% by weight of active substances, which are ofnatural origin and/or biodegradable, whereby the quantitative data refer to the weight of the cosmetic compositions without the aqueous and alcoholic solvents present in these compositions.

The composition of some preferred cosmetic compositions can be obtained from the following tables (data are given in % by weight, based on the total weight of the cleaning agent, unless otherwise stated).

| | Formulation 1 | Formulation 2 | Formulation 3 | Formulation 4 | Formulation 5 |
|---|---|---|---|---|---|
| Polylactic acid particles | 0.1 to 20 | 0.5 to 15 | 0.75 to 10 | 1.0 to 8.0 | 1.0 to 8.0 |
| Active substance b) | 0.001 to 10 | 0.01 to 7.5 | 0.05 to 5.0 | 0.05 to 5.0 | 0.1 to 3.0 |
| Misc. | To 100 | To 100 | To 100 | To 100 | To 100 |
| | Formulation 6 | Formulation 7 | Formulation 8 | Formulation 9 | Formulation 10 |
| Polylactic acid particles | 0.1 to 20 | 0.5 to 15 | 0.75 to 10 | 1.0 to 8.0 | 1.0 to 8.0 |
| Amino acid oligomer | 0.001 to 10 | 0.01 to 7.5 | 0.05 to 5.0 | 0.05 to 5.0 | 0.1 to 3.0 |
| Misc. | To 100 | To 100 | To 100 | To 100 | To 100 |
| | Formulation 11 | Formulation 12 | Formulation 13 | Formulation 14 | Formulation 15 |
| Polylactic acid particles | 0.1 to 20 | 0.5 to 15 | 0.75 to 10 | 1.0 to 8.0 | 1.0 to 8.0 |
| Quassia amara extract | 0.001 to 10 | 0.01 to 7.5 | 0.05 to 5.0 | 0.05 to 5.0 | 0.1 to 3.0 |
| Misc. | To 100 | To 100 | To 100 | To 100 | To 100 |
| | Formulation 16 | Formulation 17 | Formulation 18 | Formulation 19 | Formulation 20 |
| Polylactic acid particles | 0.1 to 20 | 0.5 to 15 | 0.75 to 10 | 1.0 to 8.0 | 1.0 to 8.0 |
| Active substance b) | 0.001 to 10 | 0.01 to 7.5 | 0.05 to 5.0 | 0.05 to 5.0 | 0.1 to 3.0 |
| Skin-conditioning active substance c) | 1.0 to 50 | 3.0 to 40 | 5.0 to 50 | 5.0 to 30 | 10 to 30 |
| Misc. | To 100 | To 100 | To 100 | To 100 | To 100 |
| | Formulation 21 | Formulation 22 | Formulation 23 | Formulation 24 | Formulation 25 |
| Polylactic acid particles | 0.1 to 20 | 0.5 to 15 | 0.75 to 10 | 1.0 to 8.0 | 1.0 to 8.0 |
| Amino acid oligomer | 0.001 to 10 | 0.01 to 7.5 | 0.05 to 5.0 | 0.05 to 5.0 | 0.1 to 3.0 |
| Skin-conditioning active substance c) | 1.0 to 50 | 3.0 to 40 | 5.0 to 50 | 5.0 to 30 | 10 to 30 |
| Misc. | To 100 | To 100 | To 100 | To 100 | To 100 |
| | Formulation 26 | Formulation 27 | Formulation 28 | Formulation 29 | Formulation 30 |
| Polylactic acid particles | 0.1 to 20 | 0.5 to 15 | 0.75 to 10 | 1.0 to 8.0 | 1.0 to 8.0 |
| Quassia amara extract | 0.001 to 10 | 0.01 to 7.5 | 0.05 to 5.0 | 0.05 to 5.0 | 0.1 to 3.0 |
| Skin-conditioning active substance c) | 1.0 to 50 | 3.0 to 40 | 5.0 to 50 | 5.0 to 30 | 10 to 30 |
| Misc. | To 100 | To 100 | To 100 | To 100 | To 100 |
| | Formulation 31 | Formulation 32 | Formulation 33 | Formulation 34 | Formulation 35 |
| Polylactic acid particles | 0.1 to 20 | 0.5 to 15 | 0.75 to 10 | 1.0 to 8.0 | 1.0 to 8.0 |
| Active substance b) | 0.001 to 10 | 0.01 to 7.5 | 0.05 to 5.0 | 0.05 to 5.0 | 0.1 to 3.0 |
| C₁₆₋₃₀ fatty acid triglyceride | 1.0 to 50 | 3.0 to 40 | 5.0 to 50 | 5.0 to 30 | 10 to 30 |
| Misc. | To 100 | To 100 | To 100 | To 100 | To 100 |
| | Formulation 36 | Formulation 37 | Formulation 38 | Formulation 39 | Formulation 40 |
| Polylactic acid particles | 0.1 to 20 | 0.5 to 15 | 0.75 to 10 | 1.0 to 8.0 | 1.0 to 8.0 |
| Active substance b) | 0.001 to 10 | 0.01 to 7.5 | 0.05 to 5.0 | 0.05 to 5.0 | 0.1 to 3.0 |
| Polyol | 0.1 to 20 | 0.5 to 15 | 1.0 to 10 | 2.0 to 8.0 | 2.0 to 8.0 |
| Misc. | To 100 | To 100 | To 100 | To 100 | To 100 |
| | Formulation 41 | Formulation 42 | Formulation 43 | Formulation 44 | Formulation 45 |
| Polylactic acid particles | 0.1 to 20 | 0.5 to 15 | 0.75 to 10 | 1.0 to 8.0 | 1.0 to 8.0 |
| Amino acid oligomer | 0.001 to 10 | 0.01 to 7.5 | 0.05 to 5.0 | 0.05 to 5.0 | 0.1 to 3.0 |
| Polyol | 0.1 to 20 | 0.5 to 15 | 1.0 to 10 | 2.0 to 8.0 | 2.0 to 8.0 |
| Misc. | To 100 | To 100 | To 100 | To 100 | To 100 |
| | Formulation 46 | Formulation 47 | Formulation 48 | Formulation 49 | Formulation 50 |
| Polylactic acid particles | 0.1 to 20 | 0.5 to 15 | 0.75 to 10 | 1.0 to 8.0 | 1.0 to 8.0 |
| Quassia amara extract | 0.001 to 10 | 0.01 to 7.5 | 0.05 to 5.0 | 0.05 to 5.0 | 0.1 to 3.0 |
| Polyol | 0.1 to 20 | 0.5 to 15 | 1.0 to 10 | 2.0 to 8.0 | 2.0 to 8.0 |
| Misc. | To 100 | To 100 | To 100 | To 100 | To 100 |
| | Formulation 51 | Formulation 52 | Formulation 53 | Formulation 54 | Formulation 55 |
| Polylactic acid particles | 0.1 to 20 | 0.5 to 15 | 0.75 to 10 | 1.0 to 8.0 | 1.0 to 8.0 |
| Active substance b) | 0.001 to 10 | 0.01 to 7.5 | 0.05 to 5.0 | 0.05 to 5.0 | 0.1 to 3.0 |
| Glycerol | 0.1 to 20 | 0.5 to 15 | 1.0 to 10 | 2.0 to 8.0 | 2.0 to 8.0 |
| Misc. | To 100 | To 100 | To 100 | To 100 | To 100 |
| | Formulation 56 | Formulation 57 | Formulation 58 | Formulation 59 | Formulation 60 |
| Polylactic acid particles | 0.1 to 20 | 0.5 to 15 | 0.75 to 10 | 1.0 to 8.0 | 1.0 to 8.0 |
| Active substance b) | 0.001 to 10 | 0.01 to 7.5 | 0.05 to 5.0 | 0.05 to 5.0 | 0.1 to 3.0 |
| Skin-conditioning active substance c) | 1.0 to 50 | 3.0 to 40 | 5.0 to 50 | 5.0 to 30 | 10 to 30 |
| Polyol | 0.1 to 20 | 0.5 to 15 | 1.0 to 10 | 2.0 to 8.0 | 2.0 to 8.0 |
| Misc. | To 100 | To 100 | To 100 | To 100 | To 100 |
| | Formulation 61 | Formulation 62 | Formulation 63 | Formulation 64 | Formulation 65 |
| Polylactic acid particles | 0.1 to 20 | 0.5 to 15 | 0.75 to 10 | 1.0 to 8.0 | 1.0 to 8.0 |
| Amino acid oligomer | 0.001 to 10 | 0.01 to 7.5 | 0.05 to 5.0 | 0.05 to 5.0 | 0.1 to 3.0 |
| Skin-conditioning active substance c) | 1.0 to 50 | 3.0 to 40 | 5.0 to 50 | 5.0 to 30 | 10 to 30 |
| Polyol | 0.1 to 20 | 0.5 to 15 | 1.0 to 10 | 2.0 to 8.0 | 2.0 to 8.0 |
| Misc. | To 100 | To 100 | To 100 | To 100 | To 100 |
| | Formulation 66 | Formulation 67 | Formulation 68 | Formulation 69 | Formulation 70 |
| Polylactic acid particles | 0.1 to 20 | 0.5 to 15 | 0.75 to 10 | 1.0 to 8.0 | 1.0 to 8.0 |
| Quassia amara extract | 0.001 to 10 | 0.01 to 7.5 | 0.05 to 5.0 | 0.05 to 5.0 | 0.1 to 3.0 |
| Skin-conditioning active substance c) | 1.0 to 50 | 3.0 to 40 | 5.0 to 50 | 5.0 to 30 | 10 to 30 |
| Polyol | 0.1 to 20 | 0.5 to 15 | 1.0 to 10 | 2.0 to 8.0 | 2.0 to 8.0 |
| Misc. | To 100 | To 100 | To 100 | To 100 | To 100 |
| | Formulation 71 | Formulation 72 | Formulation 73 | Formulation 74 | Formulation 75 |
| Polylactic acid particles | 0.1 to 20 | 0.5 to 15 | 0.75 to 10 | 1.0 to 8.0 | 1.0 to 8.0 |
| Active substance b) | 0.001 to 10 | 0.01 to 7.5 | 0.05 to 5.0 | 0.05 to 5.0 | 0.1 to 3.0 |
| C₁₆₋₃₀ fatty acid triglyceride | 1.0 to 50 | 3.0 to 40 | 5.0 to 50 | 5.0 to 30 | 10 to 30 |
| Polyol | 0.1 to 20 | 0.5 to 15 | 1.0 to 10 | 2.0 to 8.0 | 2.0 to 8.0 |
| Misc. | To 100 | To 100 | To 100 | To 100 | To 100 |
| | Formulation 76 | Formulation 77 | Formulation 78 | Formulation 79 | Formulation 80 |
| Polylactic acid particles | 0.1 to 20 | 0.5 to 15 | 0.75 to 10 | 1.0 to 8.0 | 1.0 to 8.0 |
| Active substance b) | 0.001 to 10 | 0.01 to 7.5 | 0.05 to 5.0 | 0.05 to 5.0 | 0.1 to 3.0 |
| Skin-conditioning active substance c) | 1.0 to 50 | 3.0 to 40 | 5.0 to 50 | 5.0 to 30 | 10 to 30 |
| Glycerol | 0.1 to 20 | 0.5 to 15 | 1.0 to 10 | 2.0 to 8.0 | 2.0 to 8.0 |
| Misc. | To 100 | To 100 | To 100 | To 100 | To 100 |
| | Formulation 81 | Formulation 82 | Formulation 83 | Formulation 84 | Formulation 85 |
| Polylactic acid particles | 0.1 to 20 | 0.5 to 15 | 0.75 to 10 | 1.0 to 8.0 | 1.0 to 8.0 |
| Amino acid oligomer | 0.001 to 10 | 0.01 to 7.5 | 0.05 to 5.0 | 0.05 to 5.0 | 0.1 to 3.0 |
| C₁₆₋₃₀ fatty acid triglyceride | 1.0 to 50 | 3.0 to 40 | 5.0 to 50 | 5.0 to 30 | 10 to 30 |
| Polyol | 0.1 to 20 | 0.5 to 15 | 1.0 to 10 | 2.0 to 8.0 | 2.0 to 8.0 |
| Misc. | To 100 | To 100 | To 100 | To 100 | To 100 |
| | Formulation 86 | Formulation 87 | Formulation 88 | Formulation 89 | Formulation 90 |
| Polylactic acid particles | 0.1 to 20 | 0.5 to 15 | 0.75 to 10 | 1.0 to 8.0 | 1.0 to 8.0 |
| Quassia amara extract | 0.001 to 10 | 0.01 to 7.5 | 0.05 to 5.0 | 0.05 to 5.0 | 0.1 to 3.0 |
| C₁₆₋₃₀ fatty acid triglyceride | 1.0 to 50 | 3.0 to 40 | 5.0 to 50 | 5.0 to 30 | 10 to 30 |
| Polyol | 0.1 to 20 | 0.5 to 15 | 1.0 to 10 | 2.0 to 8.0 | 2.0 to 8.0 |
| Misc. | To 100 | To 100 | To 100 | To 100 | To 100 |
| | Formulation 91 | Formulation 92 | Formulation 93 | Formulation 94 | Formulation 95 |
| Polylactic acid particles | 0.1 to 20 | 0.5 to 15 | 0.75 to 10 | 1.0 to 8.0 | 1.0 to 8.0 |
| Amino acid oligomer | 0.001 to 10 | 0.01 to 7.5 | 0.05 to 5.0 | 0.05 to 5.0 | 0.1 to 3.0 |
| C₁₆₋₃₀ fatty acid triglyceride | 1.0 to 50 | 3.0 to 40 | 5.0 to 50 | 5.0 to 30 | 10 to 30 |
| Glycerol | 0.1 to 20 | 0.5 to 15 | 1.0 to 10 | 2.0 to 8.0 | 2.0 to 8.0 |
| Misc. | To 100 | To 100 | To 100 | To 100 | To 100 |
| | Formulation 96 | Formulation 97 | Formulation 98 | Formulation 99 | Formulation 100 |
| Polylactic acid particles | 0.1 to 20 | 0.5 to 15 | 0.75 to 10 | 1.0 to 8.0 | 1.0 to 8.0 |
| Quassia amara extract | 0.001 to 10 | 0.01 to 7.5 | 0.05 to 5.0 | 0.05 to 5.0 | 0.1 to 3.0 |
| C₁₆₋₃₀ fatty acid | 1.0 to 50 | 3.0 to 40 | 5.0 to 50 | 5.0 to 30 | 10 to 30 |
| Triglyceride | | | | | |
| Glycerol | 0.1 to 20 | 0.5 to 15 | 1.0 to 10 | 2.0 to 8.0 | 2.0 to 8.0 |
| Misc. | To 100 | To 100 | To 100 | To 100 | To 100 |

Another subject of the present application is the use of the compositions of the invention for the nontherapeutic, cosmetic treatment of human skin, particularly
for treating and/or minimizing skin folds and wrinkles,
for fighting the signs of intrinsic and extrinsic skin aging, and/or
for treating tired, sagging, and/or dry skin, UV-damaged skin, and/or irritated skin.

As stated at the outset, the cosmetic compositions of the invention are characterized by an unexpected nurturing and regenerating action. A further subject of the present application therefore is a nontherapeutic method for the nontherapeutic, cosmetic treatment of human skin, particularly the treatment and/or minimizing of skin folds and wrinkles, the fighting of signs of intrinsic and extrinsic skin aging, and/or the treatment of tired, sagging, and/or dry skin, UV-damaged skin, and/or irritated skin, characterized in that a composition of the invention is applied to the skin.

The compositions of the invention can remain on the skin after application (“leave on”) or can be removed from the skin after a treatment time (“rinse off”). The treatment time of the last-mentioned products is preferably between 10 and 600 seconds, preferably between 20 and 420 seconds, and especially between 30 and 300 seconds.

### EXAMPLES

The following skin creams were prepared (quantities are given in % by weight):

| | Formulation 1 | Formulation 2 |
|---|---|---|
| Montanov ® 202 | 5.0 | 5.0 |
| Carylic/capric triglyceride | 7.0 | 7.0 |
| Cetiol CC ® | 3.0 | 3.0 |
| Coconut glyceride C12-18 | 2.0 | 2.0 |
| DC EL-8040 ID Silicone | 0.9 | 0.9 |
| Vitamin E Acetate | 0.5 | 0.5 |
| Controx ® KS C | 0.05 | 0.05 |
| Cetiol ® SB 45 | 1.5 | 1.5 |
| Water | 54.4 | 56.4 |
| Glycerol | 5.0 | 5.0 |
| Hexanediol-1,6 | 6.0 | 6.0 |
| Betafin ® BP 20 | 2.0 | 2.0 |
| Tego carbomer 140 ® | 0.4 | 0.4 |
| Ridulisse ® C GR | 0.5 | 0.5 |
| DSH-C N | 2.0 | 2.0 |
| Phenonip ® ME | 0.5 | 0.5 |
| Allantoin | 0.05 | 0.05 |
| D-Panthenol 75% | 0.75 | 0.75 |
| D,l-alpha Bisabolol | 0.05 | 0.05 |
| Quassia wood extract | — | 1.0 |
| Matrixyl ® 3000 PEG free | 3.0 | |
| Per fume | 0.35 | 0.35 |
| Simulgel ® EPG | 1.0 | 1.0 |
| Ecoscrub ® 50PC | 4.0 | 4.0 |
| Misc. | To 100 | To 100 |
| | Formulation 3 | Formulation 4 |
| Caprylic/capric triglyceride | 20.0 | 20.0 |
| Myritol ® PC | 2.5 | 2.5 |
| Coconut glyceride C12-18 | 2.5 | 2.5 |
| Cetiol Sensoft ® | 1.5 | 1.5 |
| Cetiol ® SB 45 | 1.0 | 1.0 |
| Vitamin E acetate | 0.5 | 0.5 |
| Castor oil hydrog., 40 EO | 0.2 | 0.2 |
| Water | 39.0 | 37.0 |
| Glycerol | 3.0 | 3.0 |
| EDETA BX Powder ® | 0.1 | 0.1 |
| Pemulen ® TR 1 | 0.25 | 0.25 |
| Carbopol ETD 2020 ® | 0.25 | 0.25 |
| Matrixyl ® 3000 PEG free | — | 3.0 |
| Quassia amara Wood Extract | 1.0 | — |
| NP Moist 24 | 3.0 | 3.0 |
| Euxyl ® PE 9010 | 1.0 | 1.0 |
| Orange Blossom Floral Water | 10.0 | 10.0 |
| Rose Floral Water | 10.0 | 10.0 |
| Ecoscrub ® 50PC | 4.0 | 4.0 |
| Misc. | To 100 | To 100 |

The following commercial products were used in the above tables: Montanov 202 (INCI name: Arachidyl Alcohol, Behenyl Alcohol, Arachidyl Glucoside), Seppic; Cetiol CC (INCI name: Dicaprylyl Carbonate), BASF; DC EL-8040 ID Silicone (INCI name: Isododecane, Dimethicone Crosspolymer); Dow Corning; Controx KS C (INCI name: Tocopherol, Hydrogenated Palm Glycerides Citrate), BASF; Cetiol SB 45 (INCI name: Shea Butter), BASF; Betafin BP 20 (INCI name: Betaine), Finnfeeds Finland Oy; Tego Carbomer 140 (INCI name: Carbomer), Evonik; Ridulisse C GR (INCI name: Hydrolyzed Soy Protein), Silab; DSH-C N (INCI name: Dimethylsilanol Hyaluronate), Exsymol; Phenonip ME (INCI name: Phenoxyethanol, Methylparaben, Ethylparaben) Clariant; Matrixyl 3000 PEG free (INCI name: C12-15 Alkyl Benzoate, Sorbitan Laurate, Tristearin, Acetylated Glycol Stearate, Palmitoyl Oligopeptide, Palmitoyl Tetrapeptide-7), Sederma; Simulgel EPG (INCI name: Sodium Acrylates/Sodium Aryloyldimethyl Taurate Copolymer, Polyisobutene, Caprylyl/Capryl Glucoside), Seppic; Myritol PC (INCI name: Propylene Glycol Dicaprylate/Dicaprate), BASF; Ecoscrub® 50PC (INCI name: Polylactic Acid), Micro Powders, Inc; Cetiol Sensoft (INCI name: Propylheptyl Caprylate), BASF; EDETA BX Powder (INCI name: Tetrasodium EDTA), BASF; Pemulen TR 1 (INCI name: Acrylates/C10-30 Alkyl Acrylate Crosspolymer), Lubrizol; Carbopol ETD 2020 (INCI name: Acrylates/C10-30 Alkyl Acrylate Crosspolymer), Lubrizol; NP Moist 24 (INCI name: Imperata Cylindrica Root Extract, Glycerin, Aqua (Water), Caprylyl Glycol, Carbomer, Acrylates/C 10-30 Alkyl Acrylate Crosspolymer), Sederma; Euxyl PE 9010 (INCI name: Phenoxyethanol, Ethylhexylglycerin), Schülke & Mayr.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. Cosmetic or dermatological topical compositions including, in a suitable cosmetic or dermatological carrier,
a) polylactic acid particles and
b) at least one active substance selected from the group consisting of
monomers, oligomers, and polymers of amino acids, N—C₂-C₂₄ acylamino acids, the esters and/or physiologically acceptable metal salts of said substances,
plant preparations, especially expressed juices and extracts which are obtained from plants in the group comprising Quassia amara, dill (Peucedanum graveolens), currant, cardamom (Elettaria cardamomum), black radish, butcher's broom, cinnamon, oats (Avena sativa), potato, silk, and asafoetida gum,
hyaluronic acid,
apple seed extract,
phytohormones,
isoflavonoids,
phytosterols,
triterpenoids,
tocopherols,
6,7-disubstituted 2,2-dialkyl chromanes or chromenes of general formula (II) or (III), 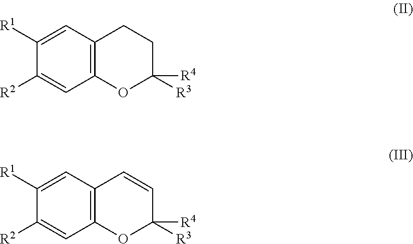
R¹ and R² representing, independently of one another, an OH group, a methoxy group, or a CF₃CH₂O group and R³ and R⁴ representing, independently of one another, a C₁-C₄ alkyl group,
ellagic acid and/or ellagates,
xanthophylls, in particular astaxanthin,
superoxide dismutases,
fermentations based on lactic acid bacteria,
ethyl hexanoate and derivatives thereof,
methyl butyrate and derivatives thereof, and
ethyl decadienoate and derivatives thereof.

2. The cosmetic compositions according to claim 1, wherein the polylactic acid particles have absolute particle sizes in the range of 1 to 1000 μm.

3. The cosmetic compositions according to claim 1, wherien, based on their weight, they include
a) 0.1 to 20% by weight polylactic acid particles, and
b) 0.001 to 10% by weight one active substance from group b).

4. The cosmetic compositions according to claim 1, wherein the active substance b) is selected from the group consisting of
oligomers of amino acids, N—C₂-C₂₄ acylamino acids, and/or the physiologically acceptable metal salts of such substances, and
extracts from leaves, bark, and/or wood of Quassia amara.

5. The cosmetic compositions according to claim 4, wherein the oligomers of amino acids, N—C₂-C₂₄ acylamino acids, and/or the physiologically acceptable metal salts of said substances are selected from the group consisting of
Tyr-Arg and its N-acylated derivatives,
Gly-His-Lys and its N-acylated derivatives,
Gly-His-Arg and its N-acylated derivatives,
Lys-Val-Lys and its N-acylated derivatives,
Val-Tyr-Val,
Gly-Gln-Pro-Arg (rigin), rigin analogues, and rigin derivatives,
Lys-Thr-Thr-Lys-Ser and its N-acylated derivatives,
Val-Gly-Val-Ala-Pro-Gly and its N-acylated derivatives,
acetyl hexapeptide-3, hexapeptide-4, hexapeptide-5, myristoyl hexapeptide-5, myristoyl hexapeptide-6, hexapeptide-8, hexapeptide-9, hexapeptide-10, L-glutamylaminoethyl indole, and
combinations of such substances.

6. The cosmetic compositions according to claim 1, further including 0.1 to 20% by weight at least one polyol selected from the group consisting of glycerol, 1,2-propylene glycol, 1,3-butylene glycol, and 1,6-hexanediol, referring to the weight of the cosmetic compositions.

7. The cosmetic compositions according to claim 1, further including 1 to 50% by weight at least one skin-conditioning active substance c) referring to the weight of the cosmetic compositions.

8. The cosmetic compositions according to claim 1, wherein they include 40 to 100% by weight of the active substances, which are of natural origin and/or biodegradable, referring to the weight of the cosmetic compositions without the aqueous and alcoholic solvents present in these compositions.

9. A nontherapeutic method for the nontherapeutic, cosmetic treatment of human skin, including the treatment and/or minimizing of skin folds and wrinkles, fighting the signs of intrinsic and extrinsic skin aging, and/or the treatment of tired, sagging, and/or dry skin, UV-damaged skin, and/or irritated skin, including applying to the skin a composition according to claim 1.
